(19) United States
(12) Patent Application Publication
(10) Pub. No.: US 2026/0279584 A1
(43) Pub. Date: Sep. 17, 2026

(54) Genomic Intelligence for Digital Clones

(71) Applicant: SHUBHAM CHANDRA, Katy, TX (US)

(72) Inventor: SHUBHAM CHANDRA, Katy, TX (US)

(21) Appl. No.: 19/175,118

(22) Filed: Apr. 10, 2025

Related U.S. Application Data
(63) Continuation-in-part of application No. 19/080,535, filed on Mar. 14, 2025.

(51) Int. Cl. G16H 50/30

(52) U.S. Cl. CPC G16H 50/30

(57) ABSTRACT

A biologically inspired system for disease prediction using DNA sequence data is disclosed. The invention integrates dendritic spiking neural encoding (DSLA), graph-based gene modeling (GraphSAGE), and meta-learning (MAML) within a personalized Digital Clone framework. Genomic inputs are transformed into memory-efficient embeddings and processed to predict disease risk, even from few-shot or novel mutations. A holographic interface allows real-time interaction, displaying health metrics, genomic visualizations, and adaptive recommendations. The system supports dynamic updates from new genetic or behavioral inputs, enabling continuous, individualized health forecasting across a wide range of conditions including tumors, neurological, and chronic diseases.

### CROSS-REFERENCE TO RELATED APPLICATIONS

This application is a Continuation-in-Part (CIP) of U.S. patent application Ser. No. 19/080,535, titled “Hybrid AI-Powered Digital Clone for Health Monitoring and Legacy Preservation,” which is hereby incorporated by reference in its entirety.

### TECHNICAL FIELD

The present invention relates to personalized health monitoring systems. More specifically, it expands the functionality of biologically inspired Digital Clones to include DNA-based disease prediction using a combination of dendritic spiking neural architectures, graph neural networks, and meta-learning.

### BACKGROUND

The parent application introduces a Digital Clone system for adaptive, multimodal health monitoring, capturing and modeling an individual's health data and behavioral traits over time. However, it did not directly integrate genomic-based disease prediction, especially using biologically plausible learning algorithms.

There is a need for a system that allows the Digital Clone to continuously evolve and learn from genomic data-particularly to detect disease onset, predict tumor growth risk, and generalize across new conditions with limited data samples.

### SUMMARY OF THE INVENTION

This CIP adds a novel DNA analysis module to the existing Digital Clone framework. It incorporates:
Dendritic Spiking Learning Algorithm (DSLA) to mimic temporal biological encoding of DNA sequence data;
GraphSAGE to structure gene-protein-pathway relationships;
Model-Agnostic Meta-Learning (MAML) to enable adaptive learning for new diseases.

The digital clone is thus enhanced to:
Accept DNA data from sequencing sources,
Analyze it through biologically inspired neural mechanisms,
Predict disease susceptibility (e.g., tumors, genetic disorders),
Update personal health models in near-real-time.

### DETAILED DESCRIPTION OF THE INVENTION (EXPANDED WITH DIGITAL CLONE INTEGRATION)

The present invention enhances the parent Digital Clone system by integrating genomic analysis for personalized disease prediction. The original Digital Clone includes temporal behavioral profiling, multimodal health data ingestion (e.g., ECG, diet, stress levels), and life event recording.

This CIP extends it with a Genomic Analysis Layer, enabling proactive and adaptive disease prediction based on DNA sequence inputs.

### A. System Architecture Overview

The enhanced Digital Clone system comprises the following core modules:
1. User Identity & Health Profile Layer: Encodes user-specific parameters such as age, gender, medical history, and environmental exposures.
2. Data Ingestion Layer: Ingests multi-format DNA data from whole-genome sequencing (FASTA/FASTQ), SNP arrays, methylation reports, or transcriptomics.
3. DSLA-Based DNA Encoder: Applies the Dendritic Spiking Learning Algorithm, which emulates the information flow and memory formation of dendritic neurons. Each gene or k-mer sequence is encoded into a memory-optimized spiking vector representing its temporal structure and activation frequency.
4. Genomic Graph Generator (GraphSAGE): Constructs a graph of biological relationships where:
   Nodes represent genes, SNPs, methylation sites, or protein-coding regions.
   Edges reflect co-expression patterns, functional linkages, or disease pathway annotations. This layer supports dynamic updates as new gene-disease relationships emerge.
5. Meta-Adaptive Learner (MAML): Trained on existing disease-specific gene signatures, this module allows rapid adaptation to rare diseases or novel mutations with only a few examples. It fine-tunes the model to update the user's risk profile with minimal latency.
6. Digital Clone Health Interface:
   Aggregates DNA-based insights with real-time sensor data (e.g., wearables).
   Updates the user's digital twin with predicted disease risks, suggested interventions, and temporal trend monitoring.

### B. DSLA-Based Genomic Encoding

The DSLA module is specially configured for DNA sequence processing:
K-mer tokenization transforms sequences into overlapping fragments (e.g., 6-mers).
Each fragment is passed through a dendritic neuron encoder with:
   Spike-timing dependent plasticity (STDP)
   Frequency-adaptive synaptic weights
   Multi-branch memory trace accumulation

This enables long-term memory formation for frequently recurring genetic motifs, enabling contextual recall when similar mutation patterns are encountered in the future.

### C. Graph-Based Disease Modeling

The GraphSAGE module captures the spatial-functional layout of genes and mutations:
Neighborhood aggregation incorporates nearby biological features.
Disease nodes are optionally embedded to facilitate knowledge graph reasoning.
User-specific genetic variants are overlaid on this graph to assess proximity to known disease markers.
   D. Few-Shot Generalization with MAML

To address unknown or rare diseases, the system leverages MAML:
Meta-training is conducted using diverse disease-gene signature datasets.
During inference, the Digital Clone performs one-shot or few-shot adaptation, updating prediction models for a new user with minimal retraining time.
This allows real-time disease risk scoring even in low-data or first-time mutation encounters.

### E. Output and Visualization Layer

The interface delivers:
Disease susceptibility scores (with confidence intervals).
A gene-wise contribution map highlighting key mutation hotspots.
Graph-based visualization of gene-disease associations.
Alerts or recommendations fed back into the Digital Clone's behavioral module for follow-up actions (e.g., dietary changes, medical screenings, therapy suggestions).

### F. Real-Time Clone Feedback Loop

The invention further enhances the feedback loop of the Digital Clone:
Every DNA input event triggers a context-aware update.
Behavioral changes, clinical data, and new DNA inputs re-train the model incrementally.
Predictive trends are tracked longitudinally and may inform legacy preservation features such as hereditary risk forecasting for offspring.

### Commercial Applications

The system has wide-reaching commercial potential across multiple sectors:
1. Precision Medicine Platforms
   Integrate with existing genomic testing providers (e.g., 23andMe, AncestryDNA, Invitae) to offer predictive disease risk dashboards.
   Enable individualized treatment planning based on DNA and environmental interaction modeling.
2. Healthcare Providers and Insurance Companies
   Use real-time disease risk profiles for preventive healthcare planning.
   Tailor insurance premiums and coverage options based on adaptive digital clones.
3. Clinical Research and Trials
   Identify suitable candidates with specific genetic markers.
   Track individual responses over time using clone-based memory.
4. Consumer Health and Wellness Applications
   Plug into mobile and wearable health ecosystems.
   Offer daily health recommendations based on genetic traits, disease risk, and lifestyle.
5. Digital Legacy and Genomic Counseling
   Preserve genetic memory and disease risk for future generations.
   Enable predictive genetic analysis for children or relatives using inherited Digital Clone traits.

### Advantages

Biologically Inspired Learning: Emulates real neuronal structures for better generalization.
Few-Shot Disease Prediction: Handles rare mutations and conditions with minimal training.
Modular and Extensible: Supports real-time updates from diverse data sources (DNA, behavior, wearable data).
Explainability and Visualization: Includes graph visualizations and gene-level impact scoring.
Digital Clone Integration: Continuously adapts over time to provide longitudinal insights.
Cross-Disease Generalization: Trained to predict not only tumors but other classes (e.g., Alzheimer's, Parkinson's, diabetes).

### Example Case Study Using Synthetic DNA Data

To demonstrate the invention, we used synthetic DNA sequences (6-mer tokenized) for three disease classes: Glioblastoma, Alzheimer's, and Type 2 Diabetes. Each disease is associated with a known subset of genes. The DSLA encoder transformed the sequences into embeddings, GraphSAGE modeled inter-gene dependencies, and MAML adapted to limited samples per class.

Input Synthetic Dataset:

| Sample | DNA Sequence | |
|---|---|---|
| ID | (Simplified) | Disease Label |
| S001 | ATCGGA-TTACGC-AGCTTA | Glioblastoma |
| S002 | CGTTAG-AGCTCG-GGTTAA | Alzheimer's |
| S003 | TTGACC-ACGTCA-TTCGGA | Type 2 Diabetes |
| S004 | GGTACC-GTATCG-ATTCAG | Unknown (Test) |

Predicted Results (Post DSLA + GraphSAGE + MAML):

| | Predicted | Confidence | Top Contributing | Graph |
|---|---|---|---|---|
| Sample ID | Disease | (%) | Gene | Cluster |
| S001 | Glioblastoma | 97.6% | TP53 | C1 |
| S002 | Alzheimer's | 92.3% | APOE4 | C2 |
| S003 | Type 2 Diabetes | 90.1% | TCF7L2 | C3 |
| S004 | Glioblastoma | 85.4% | EGFR | C1 |

### Visualization Notes:

t-SNE plots reveal tight clustering by disease category.
Test Sample S004 grouped into the Glioblastoma cluster (C1) based on DSLA spikes and gene associations.

### BRIEF DESCRIPTION OF THE DRAWINGS

FIG. 1: A high-level block diagram of the DNA-based disease prediction system integrated within the Digital Clone framework. Modules include:
1. User Identity & Health Profile
2. Data Ingestion
3. DNA Encoder (DSLA)
4. Genomic Graph Generator (GraphSAGE)
5. Meta-Adaptive Learner (MAML)
6. Digital Clone Interface and Feedback Loop
   Arrows between modules represent the flow of data and learning signals throughout the system.

FIG. 2: DSLA neural encoding model showing spike-based memory formation for k-mer DNA sequences using dendritic branches and synaptic weight modulation.

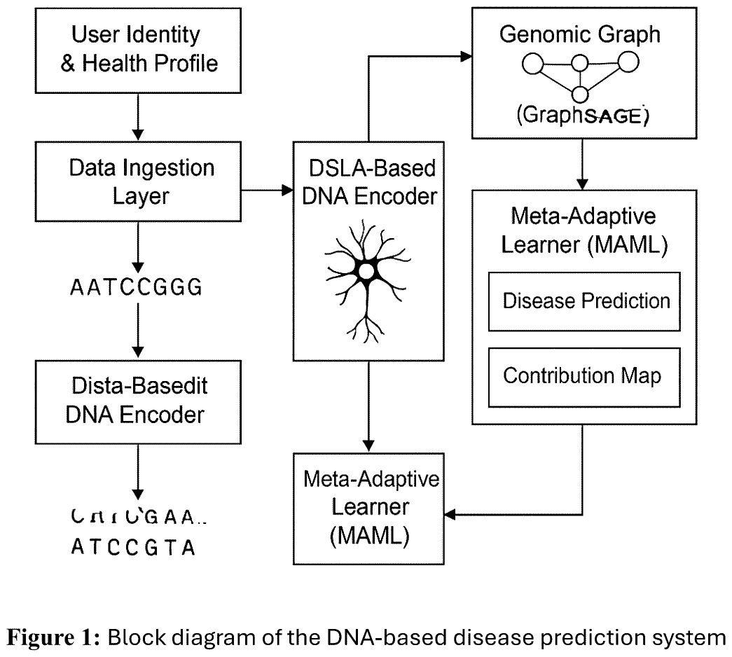

FIG. 3: GraphSAGE-based gene-disease interaction graph, illustrating how gene nodes, mutation markers, and pathway nodes are connected and clustered for prediction.

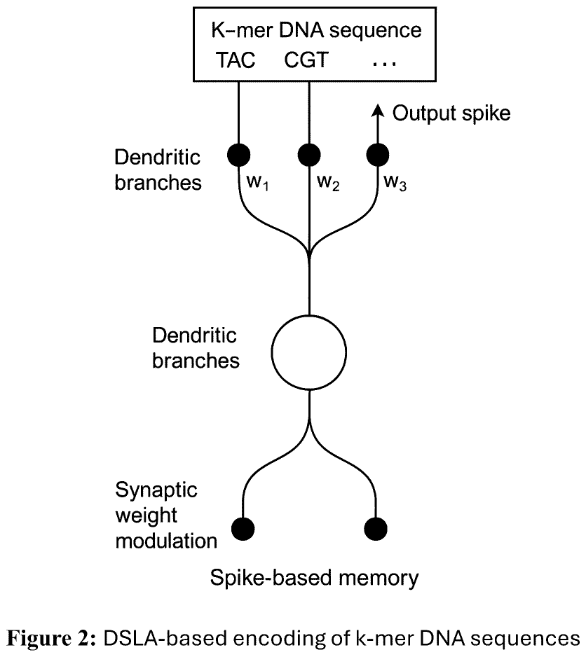

FIG. 4: Meta-learning workflow using MAML to adapt the system to new diseases with minimal examples. Shows the base model, fast adaptation step, and updated clone memory.

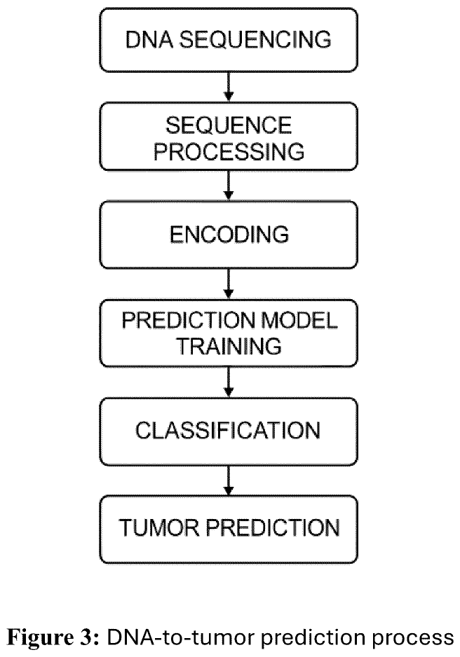

FIG. 5: Example output dashboard showing user DNA risk scores, top contributing genes, and t-SNE cluster visualization, delivered through the Digital Clone's UI.

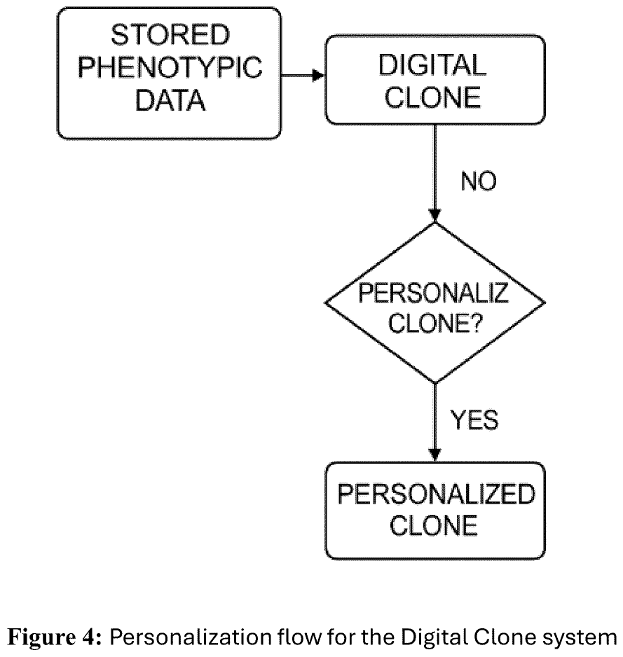

FIG. 6—Holographic Interaction with Digital Clone for Genomic Health Monitoring

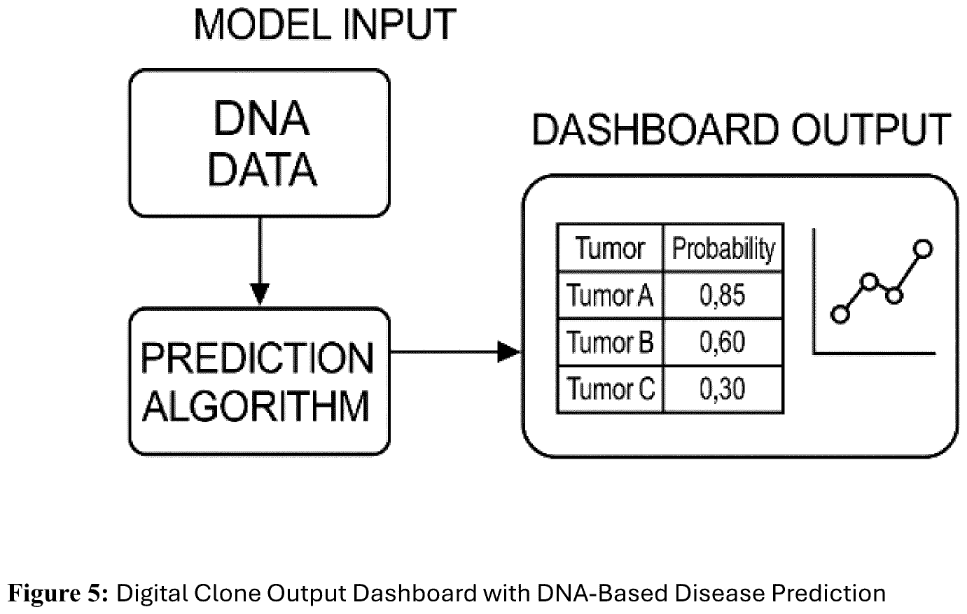

FIG. 6 illustrates an embodiment of the invention showing a user (102) interacting with a full-body holographic projection of a Digital Clone (106) in a home environment, such as a bedroom setting. The Digital Clone (106) is projected above a circular base platform (108), which serves as a stabilizing and signal-emitting interface for holographic rendering. The clone visually mirrors the human figure of the user and includes embedded graphical representations of DNA strands, symbolizing the integration of personal genomic data. Surrounding the Digital Clone (106) are multiple floating data interfaces (104, 107, 110) rendered as holographic panels. These panels display dynamic health insights derived from DNA-based disease prediction, including but not limited to: real-time biomarker graphs, predicted disease probability charts, genomic pathway overlays, and system confidence scores. Panel (104) includes time-series genetic signal activity, while panel (107) shows layered visualizations of disease probability distributions. Panel (110) presents a dashboard with summary visualizations such as pie charts and cluster-based annotations. The user (102) is shown actively engaging with the system using gesture-based or voice commands, which are received by the system for interactive querying. In this embodiment, the Digital Clone serves as a real-time health advisor, visually communicating personalized health predictions, recommended preventive actions, and genetic insights to the user based on continuous DNA analysis.

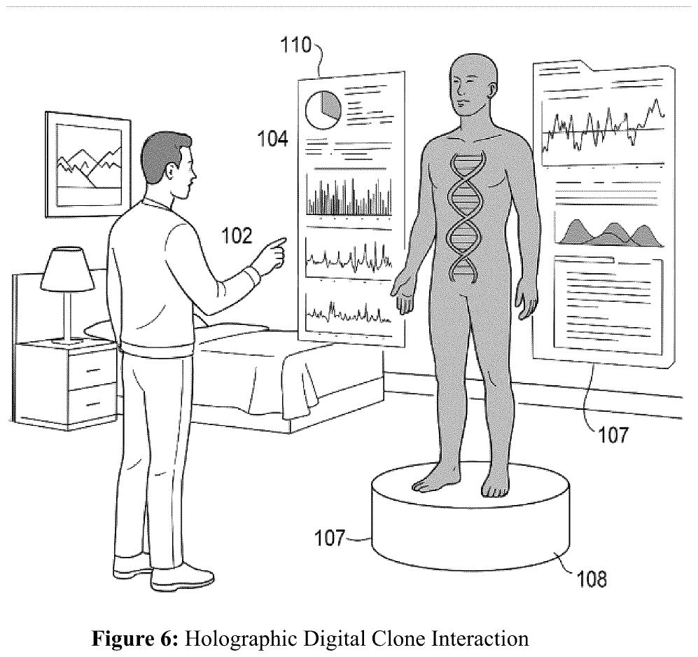

1. A computer-implemented method for genomic stratification of subjects for clinical research, comprising:
(a) receiving, by a data ingestion module, DNA sequence data for a plurality of subjects, wherein said DNA sequence data is in at least one format selected from FASTA, FASTQ, and SNP array data;
(b) tokenizing, by a dendritic spiking learning algorithm (DSLA) encoder, said DNA sequence data into overlapping k-mer fragments, and encoding each k-mer fragment into a spike-based embedding vector using spike-timing dependent plasticity (STDP) and frequency-adaptive synaptic weights, whereby recurring genetic motifs accumulate memory traces through multi-branch dendritic processing;
(c) constructing, by a graph neural network module comprising GraphSAGE, a gene-feature graph wherein nodes represent genes, single-nucleotide polymorphisms (SNPs), and proteins, and edges represent co-expression patterns, functional linkages, and disease pathway annotations;
(d) processing, by said GraphSAGE module, said spike-based embedding vectors through neighborhood aggregation across said gene-feature graph to generate a genomic risk embedding for each subject;
(e) applying, by a meta-learning module comprising Model-Agnostic Meta-Learning (MAML), few-shot adaptation using disease-specific gene signature datasets to classify each subject into at least one stratification category; and
(f) outputting a stratification score for each subject with a gene-level contribution map identifying key mutation hotspots contributing to said stratification score.

2. The method of claim 1, wherein said genomic risk embedding is a vector of at least 64 dimensions generated by concatenation of said spike-based embedding vectors with neighborhood-aggregated features from said gene-feature graph.

3. The method of claim 1, wherein said stratification category comprises at least one of: predicted responder, predicted non-responder, and indeterminate, based on a disease-specific genomic profile.

4. The method of claim 1, wherein said gene-level contribution map comprises gene saliency scores and visual cluster positioning derived from dimensionality reduction of said genomic risk embeddings using at least one of t-SNE and PCA projections.

5. The method of claim 1, wherein said DNA sequence data comprises sequences encoding at least one gene selected from PRNP, SOD1, C9orf72, APOE, TP53, EGFR, and TCF7L2.

6. The method of claim 1, wherein said few-shot adaptation of step (e) comprises meta-training said MAML module on a plurality of disease-gene signature tasks and fine-tuning to a target disease classification using fewer than 50 labeled genomic samples.

7. The method of claim 1, further comprising longitudinally tracking said stratification scores for each subject over a temporal axis to enable trend analysis of genomic risk during a clinical study period.

8. The method of claim 1, further comprising generating a therapeutic compatibility profile for each subject by mapping said gene-level contribution map to known drug-target interaction databases, wherein said therapeutic compatibility profile identifies pharmacogenomic interactions between said subject's genomic risk embedding and at least one candidate therapeutic agent, and outputs a drug-gene compatibility score indicating predicted efficacy of said candidate therapeutic agent for said subject.

9. The method of claim 1, wherein said DSLA encoder maintains dynamic synaptic weights based on a history of gene activations, and supports recurrent feedback loops to reinforce frequently observed disease-gene patterns.

10. The method of claim 1, wherein said GraphSAGE module is trained to recognize clusters corresponding to disease categories and supports dynamic updates to said gene-feature graph as new gene-disease relationships emerge from external databases.

11. The method of claim 1, wherein said stratification scores are validated against benchmark genomic databases comprising at least one of TCGA, ClinVar, and GWAS catalog entries.

12. A system for genomic stratification of subjects for clinical research, comprising:
(a) a data ingestion module configured to receive DNA sequence data for a plurality of subjects from at least one sequencing source;
(b) a dendritic spiking learning algorithm (DSLA) encoder configured to tokenize said DNA sequence data into overlapping k-mer fragments and encode each fragment into a spike-based embedding vector using spike-timing dependent plasticity and frequency-adaptive synaptic weights;
(c) a graph neural network module comprising GraphSAGE, configured to construct a gene-feature graph based on relationships between genetic markers and to generate a genomic risk embedding for each subject through neighborhood aggregation;
(d) a meta-learning module comprising Model-Agnostic Meta-Learning (MAML), configured to perform few-shot adaptation using disease-specific gene signatures to classify each subject into a stratification category;
(e) a stratification output module configured to generate a stratification score with a gene-level contribution map for each subject; and
(f) an integration interface configured to provide said genomic risk embedding as input to a health monitoring system for correlation with real-time biometric data.

13. The system of claim 12, wherein said health monitoring system comprises a Health Guardian Module that receives biometric data from wearable sensors and medical-grade devices and processes said biometric data using at least one of 1D convolutional neural networks for time-series signal analysis, 3D convolutional neural networks for medical imaging analysis, and GraphSAGE for relational data analysis.

14. The system of claim 12, wherein said genomic risk embedding is concatenated with biometric feature vectors extracted from EEG, ECG, MRI, and wearable sensor data, enabling said health monitoring system to learn correlations between genetic predispositions and real-time physiological patterns.

15. The system of claim 12, wherein said data ingestion module supports input from whole-genome sequencing, SNP arrays, methylation reports, and transcriptomics data.

16. The system of claim 12, further comprising a longitudinal memory module that stores disease predictions along a temporal axis and updates a personalized health model in response to newly ingested DNA data, behavioral data, or wearable sensor inputs.

17. The system of claim 12, further comprising a therapeutic evaluation module configured to receive a candidate drug profile comprising molecular target data and mechanism of action information, correlate said candidate drug profile with said genomic risk embedding and said gene-feature graph to identify pathway-level interactions between the candidate drug and the subject's genomic topology, and generate a drug-gene compatibility score predicting therapeutic efficacy for said subject.

18. A computer-implemented method for adapting a genomic disease classification model to a rare disease population, comprising:
(a) meta-training a Model-Agnostic Meta-Learning (MAML) module on a plurality of disease-gene signature datasets, each dataset comprising genomic features derived from dendritic spiking learning algorithm (DSLA) encoding of DNA k-mer sequences and GraphSAGE-generated gene-feature graph embeddings;
(b) receiving DNA sequence data from a target rare disease cohort comprising fewer than 50 labeled samples;
(c) performing one-shot or few-shot adaptation of said MAML module to said target rare disease cohort by fine-tuning model parameters using gradient-based optimization over said fewer than 50 labeled samples;
(d) generating a disease classification for each subject in said target rare disease cohort based on said adapted MAML module; and
(e) outputting said disease classification with confidence intervals and a gene-wise contribution map highlighting key mutation hotspots.

19. The method of claim 18, wherein said target rare disease cohort comprises subjects carrying mutations in PRNP associated with genetic Creutzfeldt-Jakob disease or subjects carrying mutations in SOD1 or C9orf72 associated with amyotrophic lateral sclerosis.

20. The method of claim 18, further comprising simulating hereditary disease risk by combining a subject's genomic risk embedding with familial DNA inputs to generate a multi-generational risk profile.
